(19) United States
(12) Patent Application Publication
(10) Pub. No.: US 2026/0233025 A1
(43) Pub. Date: Aug. 13, 2026

(54) ELECTRONIC MEDICINE FOR BIOMEDICAL APPLICATION AND METHOD FOR MANUFACTURING THE SAME

(71) Applicant: CHUNGBUK NATIONAL UNIVERSITY INDUSTRY-ACADEMIC COOPERATION FOUNDATION, Chungcheongbuk-do (KR)

(72) Inventor: Jeong Hyun KWON, Sejong-si (KR)

(21) Appl. No.: 19/436,241

(22) Filed: Dec. 30, 2025

(30) Foreign Application Priority Data
Feb. 12, 2025 (KR) 10-2025-0017702

(51) Int. Cl. A61N 5/06

(52) U.S. Cl. CPC A61N 5/062; A61N 2005/063; A61N 2005/0632; A61N 2005/0653; A61N 2005/0663

(57) ABSTRACT

The present disclosure relates to an electronic medicine. The electronic medicine includes a fiber, a light-emitting element wound around the fiber, and an encapsulation part, which is an encapsulation barrier configured to encapsulate a surface of the light-emitting element. According to the present disclosure, since a light output intensity increases according to the number of times of rolling, various biomedical applications may be possible through in vivo attachment and in vivo insertion that require high power.

### CROSS-REFERENCE TO RELATED APPLICATION

This application claims priority to Korean Patent Application No. 10-2025-0017702 filed on February 12, 2025 and all the benefits accruing therefrom under 35 U.S.C. §119, the contents of which are incorporated by reference in their entirety.

### FIELD

The present disclosure relates to an electronic medicine, and more particularly, to an ultra-high efficiency bio-light emitting element fiber electronic medicine capable of being attached to and inserted into a body for biomedical applications.

### BACKGROUND

Electronic medicines refer to new concepts of therapeutic devices or systems that treat diseases or alleviate symptoms through integration of electronic technologies and medical sciences. Instead of traditional medicine therapy, electronic medicines use electrical signals, stimulation, ultrasound, etc., to induce physiological changes in the body so as to provide therapeutic effects.

There are deep-brain stimulators used for treatment of Parkinson’s disease, depression, and obsessive-compulsive disorder, vagus nerve stimulators used for treatment of epilepsy and depression, cardiac pacemakers used for treatment of cardiovascular diseases such as arrhythmias, muscle stimulators utilized for rehabilitation therapy or recovery of exercise functions, skin-adhesive patches used for pain management, sleep improvement, smoking cessation assistance, etc., and ultrasonic and optical stimulation devices.

The existing low level laser therapy (LLLT) methods such as laser or light-emitting diode (LED) light therapies are based on a plurality of hard-point light sources, resulting in uneven light irradiation, very complex, and bulky, thereby restricting their use in treatment sites and portability. In addition, it has a significant limitation in that it is not inserted into or attached to the human body due to issues such as treatment uniformity and heat generation.

In various physical stimulus sources such as light, sound, and electromagnetic fields, light stimulation is considered as the most biocompatible stimulation source, and various light therapy researches are being actively carried out for wound healing, skin beauty, pain relief, Alzheimer’s disease, Parkinson’s disease, etc., at home and abroad. However, most current light therapy devices use point light sources such as the lasers or light-emitting diode (LED) light sources to lead to uneven light irradiation and localized heating of the irradiated areas. On the other hand, when using the organic light-emitting diode (OLED) light source, it is possible to produce a treatment agent on various substrates such as fibers, glass, textiles, plastics, etc. In addition, an emission wavelength and transparency may be adjusted according to a structural design to make it easy to configure the treatment agent in a variety of platform shapes such as the insert-type and attachment-type.

For direct deposition-type displays, solution process-based fiber displays have been widely reported, but there is a limitation that it is difficult to ensure driving lifespan and reliability due to limitations such as impurity exposure. Another method that is a thermal vacuum deposition process, was expected to be able to reduce an impurity content and thus solve the problem of low reliability. However, it has a limitation such as requirement for an additional pattern mask and low manufacturing yield. As a result, the direct deposition-type fiber displays have very low electro-optic performance, and their reliability also fails to satisfy levels required by the displays. When depositing elements on a fiber having a cylindrical and special structure, a low step coverage during the process causes instability of the devices to cause a limitation in their use as OLED light patches. Thus, when there are inherent limitations in performance and characteristics of the display light source itself, it is obvious that technology based on such the light source will ultimately result in a limited form factor.

FIG. 1 illustrates a conventional high-performance phosphorescent RGB fiber-type OLED.

Referring to FIG. 1, a solution-based high-performance phosphorescent RGB fiber-type OLED is illustrated using a conventional deep coating method. This technology demonstrated the highest level of efficiency in the world, with luminance of up to about 10,000 nits or more and efficiency of about 60 cd/A or more, based on green phosphor devices, and the fiber-type OLED are woven in a 4×4 array into textiles to implement an OLED passive matrix. This technology is based on a solution process, which is simple in production. However, it has issues such as low brightness, short lifespan, and reliability, which make it difficult to be utilized for photodynamic therapy (PDT), and there is also a limitation with a difficulty of achieving top emission from the fibers.

FIG. 2 is a view illustrating a conventional high-performance phosphorescent RGB fiber-type OLED.

Referring to FIG. 2, this is a technology that is deviated from the conventional solution process of the deep coating to apply a vacuum deposition method in the production of the fiber OLEDs. The vacuum deposition-based fiber-type OLED exhibits brightness of about 59335 cd/m², efficiency of about 70.89 cd/A, and an operation lifespan of about 720h, thereby improving the efficiency and lifespan compared to the conventional solution-process fiber OLED. However, this requires a very delicate process involving the use of a shadow mask based on the vacuum deposition, and yield due to the production is low to reduce possibility of commercialization. In addition, it is difficult to achieve the uniform emission across the entire fiber, and there are issues with a low output and short lifespan due to a PDT effect.

FIG. 3 is a view illustrating an example of using a conventional implantable LED light source.

Referring to FIG. 3, in the treatment using the conventional implantable LED light source, only a specific local area is exposed to generate heat due to the point light source.

Although light-based therapies are mainly used by irradiating light onto affected areas in an external environment such as for wound healing and the treatment of jaundice in newborns, the human diseases often originate from internal organs. Until now, there has been almost no research on organic-matrix light sources for implantable biomedical applications. Thus, various issues associated with the existing light sources (such as halogen lamps, lasers, LEDs, etc.), such as miniaturization, simplification, and costs, may be resolved by applying flexible OLED light sources. In particular, the existing implantable LED light sources have the limitations of being unable to stimulate only specific local areas and having difficulty in overcoming the limitations of high heat generation.

Photodynamic therapy (PDT), which is applied as a treatment method for selectively killing cancer cells or bacteria, requires strong light of about 100 mW/cm². Thus, since a maximum output of the current OLEDs or QLEDs is less than about 15 mW/cm², there are limitations in using the OLEDs or QLEDs as light sources for biomedical applications when operating at their maximum output, as the light sources exhibit rapid degradation in lifespan.

As described above, the existing LED-based implantable light sources have been criticized for providing the uneven light irradiation to very limited local areas and for generating internal heat. Thus, there is a need for a design of an ultra-high efficiency organic light source platform that is capable of being inserted into the body and applied in all fields of biomedicine, as well as the development of related original technologies.

In addition, as a size of an object increases, a thickness of its skin layer also increases to make it difficult to target light irradiation to internal organs. Thus, it is considered that the possibility of the treatment would be greater if light could be directly delivered by inserting the light source into the body. As a result, there is a need to develop a method for more intuitively and effectively delivering the light into the body by directly inserting the light source for irradiation so as to treat a target organ. Particularly, glioblastoma is a highly malignant and intractable disease that progresses rapidly among primary brain tumors. If left untreated, glioblastoma leads to death within about 3 months to about 6 months, and even though the use of all available treatment methods, an average survival period is only 12-14 months. In addition, due to a lack of effective treatment methods developed over the past 20 years, a survival rate remains low. Therefore, there is a need for the design and development of the biocompatible and implantable bio-organic light source structures that are capable of being inserted in various shapes and sizes while uniformly delivering high-power light sources over a certain area in all directions.

### Prior Art Document

### Patent Document

Korean Patent Registration No. 10-2382643

### SUMMARY

The present disclosure provides a new form factor for biomedical applications to effectively apply OLED light sources, which have their own unique characteristics and have been industrially verified, to the form factor, thereby providing a next-generation implantable OLED form factor that has not been developed previously.

The object of the present disclosure are not limited to those mentioned above, and other objects that are not mentioned will be clearly understood by those skilled in the art from the following description.

According to an exemplary embodiment, an electronic medicine includes: a fiber; a light-emitting element wound around the fiber; and an encapsulation part, which is an encapsulation barrier configured to encapsulate a surface of the light-emitting element.

An adhesive may be applied between the fiber and the light-emitting element.

The light-emitting element may be provided in the form of a patch, the adhesive may be applied to one surface of the light-emitting element, and the light-emitting element may be wound around the fiber in a manner, in which the fiber is rolled from one end to the other end of the one surface of the light-emitting element, to which the adhesive is applied.

The light emitting-element may include an organic light-emitting diode (OLED).

The encapsulation part may include: an inorganic layer disposed on the light-emitting element and made of an inorganic material; a polymer layer disposed on the inorganic layer and made of a polymer material; and an organic layer disposed on the polymer layer and made of an organic material.

The encapsulation layer may be provided by crossing one or more inorganic layers and one or more polymer layers.

The organic layer may be made of parylene-C.

According to another exemplary embodiment, a method for manufacturing an electronic medicine includes: applying an adhesive to one surface of a patch-type light-emitting element; positioning a fiber at one end of one surface of the light-emitting element, to which the adhesive is applied, to perform rolling while winding the fiber up to the other end of the one surface of the light-emitting element; and forming an encapsulation part on the other surface of the light-emitting element.

The light emitting-element may include an organic light-emitting diode (OLED).

The forming of the encapsulation part may include: forming an inorganic layer made of an inorganic material on the light-emitting element; forming a polymer layer made of a polymer material on the inorganic layer; and forming an organic layer made of an organic material on the polymer layer.

In the forming of the encapsulation part, one or more inorganic layers and one or more polymer layers may be formed to cross each other.

The organic layer may be made of parylene-C.

### BRIEF DESCRIPTION OF THE DRAWINGS

Exemplary embodiments can be understood in more detail from the following description taken in conjunction with the accompanying drawings, in which:

FIG. 1 illustrates a conventional high-performance phosphorescent RGB fiber-type OLED;

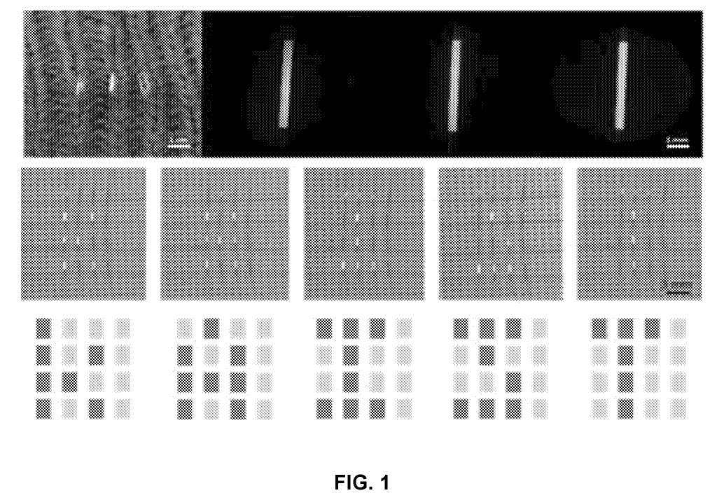

FIG. 2 is a view illustrating a conventional high-performance phosphorescent RGB fiber-type OLED;

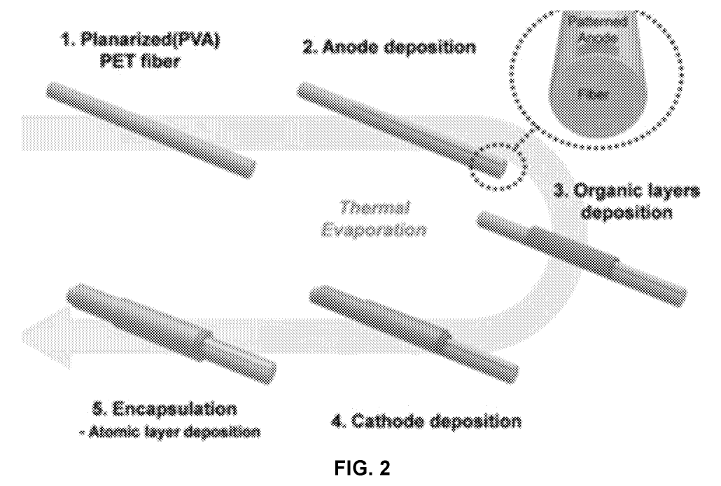

FIG. 3 is a view illustrating an example of using a conventional implantable LED light source;

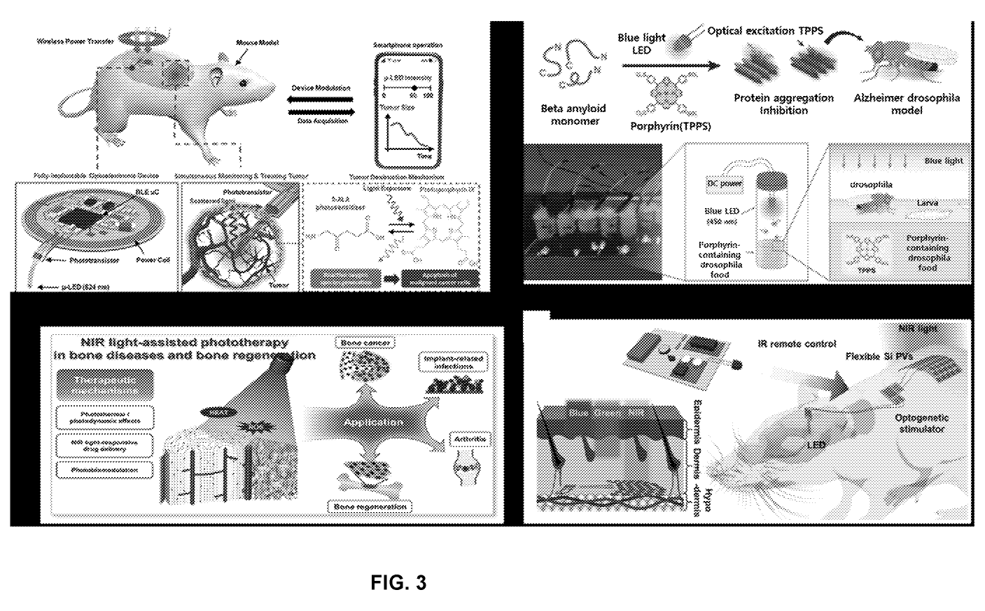

FIG. 4 is a view illustrating a cross-sectional structure of an electronic medicine according to an embodiment;

FIG. 5 illustrates a structure of an encapsulation part according to an embodiment of the present disclosure;

FIG. 6 is a flowchart illustrating a method for manufacturing an electronic medicine according to an embodiment;

FIG. 7 is a flowchart illustrating a process of forming an encapsulation part according to an embodiment;

FIG. 8 is an exemplary view for explaining a process of winding a light-emitting element on a fiber according to an embodiment;

FIG. 9 is an exemplary view for explaining a light output structure in the electronic medicine according to an embodiment; and

FIG. 10 is a graph showing results of simulating a change in light output depending on the number of times of rolling according to an embodiment.

### DETAILED DESCRIPTION

Since the present disclosure may have diverse modified embodiments, specific embodiments are illustrated in the drawings and are described in detail. However, this does not limit the present disclosure within specific embodiments and it should be understood that the present disclosure covers all the modifications, equivalents, and replacements within the idea and technical scope of the present disclosure.

In the following description, the technical terms are used only for explaining a specific exemplary embodiment while not limiting the present disclosure. The terms of a singular form may include plural forms unless referred to the contrary. In this specification, it should be understood that the terms such as “comprise/include” or “have” are intended to designate the presence of features, numbers, steps, operations, components, parts, or combinations thereof described in the specification, but does not exclude in advance the possibility of the existence or addition of elements, numbers, steps, operations, components, parts, or combinations thereof.

Unless defined otherwise, all terms used herein, including technical and scientific terms, have the same meaning as commonly understood by one of ordinary skill in the art to which this invention belongs. Terms such as terms that are generally used and have been in dictionaries should be construed as having meanings matched with contextual meanings in the art. In this description, unless defined clearly, terms are not ideally, excessively construed as formal meanings.

In addition, when describing with reference to the accompanying drawings, identical components will be assigned the same reference numerals regardless of the reference numerals, and overlapping descriptions thereof will be omitted. Moreover, detailed descriptions related to well-known functions or configurations will be ruled out in order not to unnecessarily obscure subject matters of the present disclosure.

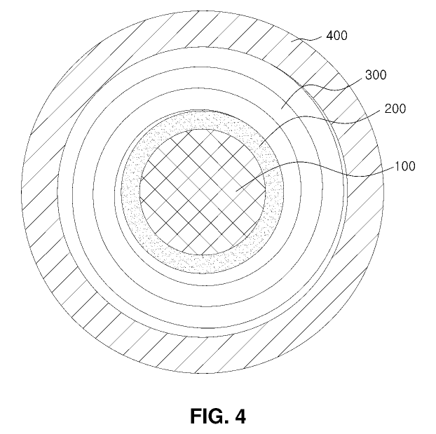

FIG. 4 is a view illustrating a cross-sectional structure of an electronic medicine according to an embodiment.

Referring to FIG. 4, an electronic medicine according to an embodiment of the present disclosure includes a fiber 100, an adhesive 200, a light-emitting element 300, and an encapsulation part 400.

In the present disclosure, the form of the fiber 100 may be variously implemented. That is, a cross-sectional shape of the fiber 100 is not limited to a circular shape and may be implemented in various shapes such as square, triangular, etc.

The light-emitting element 300 is wound around the fiber 100.

The adhesive 200 may be applied between the fiber 100 and the light-emitting element 300. In an embodiment of the present disclosure, the adhesive 200 may be a thermosetting adhesive.

The encapsulation part 400 may be an encapsulation barrier for encapsulating a surface of the light-emitting element 300.

In an embodiment of the present disclosure, the light-emitting element 300 may be in the form of a patch. Here, the adhesive 200 may be applied to one surface of the light-emitting element 300, and the light-emitting element 300 may be wound around the fiber 100 by rolling the fiber 100 from one end to the other end of the one surface of the light-emitting element 300, to which the adhesive 200 is applied.

In an embodiment of the present disclosure, a biocompatible thermosetting adhesive may be applied to one surface of the light-emitting element 300 in a spin-coating manner.

The light-emitting element 300 may be an organic light-emitting diode (OLED). In an embodiment of the present disclosure, the light-emitting element 300 may be implemented as a transparent flexible OLED. In addition, in another embodiment of the present disclosure, the light-emitting element may be implemented using various light sources such as QLEDs and QD-OLEDs, and thus, optical fibers capable of emitting light having various wavelengths including infrared rays and ultraviolet rays may be implemented.

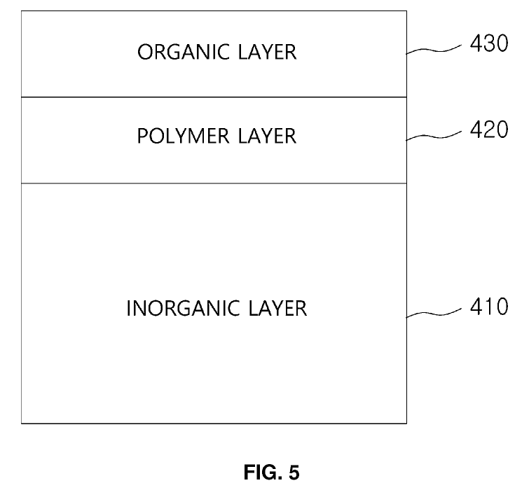

FIG. 5 illustrates a structure of the encapsulation part according to an embodiment of the present disclosure.

Referring to FIG. 5, the encapsulation part 400 may be disposed on the light-emitting element 300 and may include an inorganic layer 410 made of an inorganic material, a polymer layer 420 disposed on the inorganic layer 410 and made of a polymer material, and an organic layer 430 disposed on the polymer layer 420 and made of an organic material.

The encapsulation part 400 may be disposed by crossing one or more inorganic layers and one or more polymer layers.

In an embodiment of the present disclosure, the inorganic layer 410 may be implemented as a nanolaminate (AT NL) in which an Al₂O₃ thin film and a TiO₂ thin film are deposited to cross each other.

In an embodiment of the present disclosure, the polymer layer 420 may be made of a silamer.

The organic layer 430 may be made of parylene-C.

In the present disclosure, to insert the OLED fiber into the body, a biocompatible encapsulation packaging technology may be applied. Here, a multifunctional encapsulation film technology that satisfies all of the following properties of high transmittance, high flexibility, a water vapor transmission rate (WVTR) of about 10⁻⁶ g/m²/day, and high water resistance may be applied.

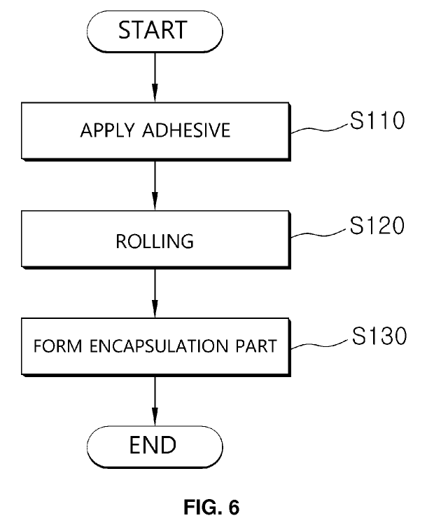

FIG. 6 is a flowchart illustrating a method for manufacturing an electronic medicine according to an embodiment.

Referring to FIG. 6, a method for manufacturing an electronic medicine according to the present disclosure includes: a process (S110) of applying an adhesive to one surface of a light-emitting element in the form of a patch, a process (S120) of positioning a fiber at one end of the one surface of the light-emitting element, to which the adhesive is applied, a process (S120) of rolling the fiber while winding the fiber to the other end of the one surface of the light-emitting element, and a process (S130) of forming an encapsulation part on the other surface of the light-emitting element.

The light-emitting element according to an embodiment of the present disclosure may be an organic light-emitting diode (OLED).

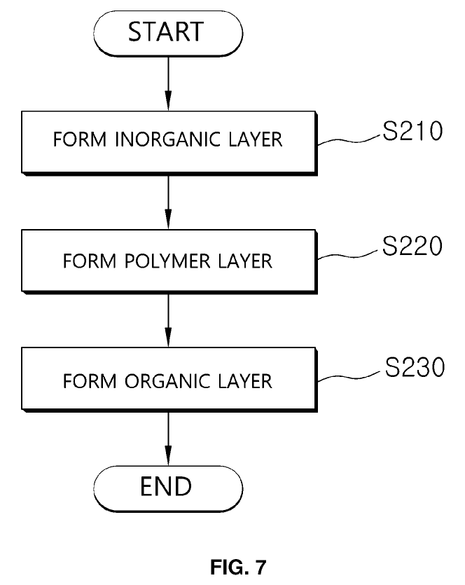

FIG. 7 is a flowchart illustrating the process of forming the encapsulation part according to an embodiment.

Referring to FIG. 7, the process (S130) of forming the encapsulation part may include a process (210) of forming an inorganic layer made of an inorganic material on the light-emitting element, a process (S220) of forming a polymer layer made of a polymer material on the inorganic layer, and a process (S230) of forming an organic layer made of an organic material on the polymer layer.

In an embodiment of the present disclosure, in the process (S130) of forming the encapsulation part, one or more inorganic layers and one or more polymer layers may be formed to cross each other.

In an embodiment of the present disclosure, the organic layer may be made of parylene-C.

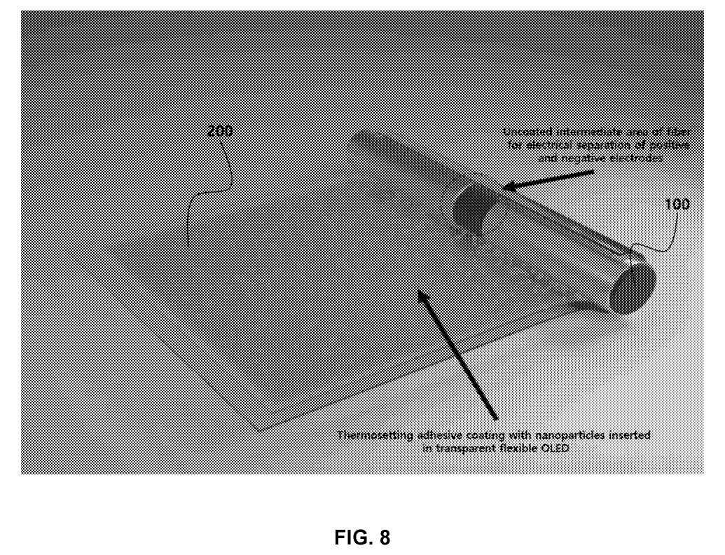

FIG. 8 is an exemplary view for explaining a process of winding the light-emitting element on the fiber according to an embodiment.

In the example of FIG. 8, the fiber 100 may be made of a conductive material and be coated with metal throughout. However, to electrically separate positive and negative electrodes from each other, an intermediate portion of the fiber 100 may not be coated with a metal, and the light-emitting element 300 may be implemented as a transparent flexible OLED. More specifically, the rolling may be performed in a state in which the positive electrode and negative electrode of the transparent flexible OLED are in contact with a conductive area of the fiber 100. That is, in an embodiment of the present disclosure, the fiber 100, in which the area in contact with the positive electrode and the negative electrode of the OLED is made of a conductive material, and the intermediate portion has a non-conductive area, may be manufactured. Alternatively, in another embodiment of the present disclosure, a conductive material having a light reflective function, in addition to the metal, may be applied on a non-conductive fiber to manufacture the fiber. Here, when applying the conductive material on the fiber, various methods such as solution processes (printing processes, dip coating processes, etc.) or vacuum deposition may be used for manufacturing.

In addition, the biocompatible thermosetting adhesive 200 into which nanoparticles are inserted may be applied to one surface of the light-emitting element 300 in a spin-coating manner. In this manner, the nanoparticles of various refractive indexes and sizes such as SiO₂ and TiO₂ into the adhesive 200 may be inserted to maximize light extraction due to a light scattering effect.

The fiber 100 may be disposed at one end of one surface of the light-emitting element 300, to which the adhesive 200 is applied. The fiber 100 may be rolled while being wound around the other end of the one surface of the light-emitting element 300, while being in contact with the fiber 100 with the anode and the cathode (positive and negative electrodes). Here, an effective rolling operation may be possible due to adhesion of the adhesive layer 200, and an air gap (refractive index of about 1) generated by the rolling may be filled with a thermosetting adhesive (refractive index of about 1.5 or higher) to suppress a light loss.

In the present disclosure, the fiber 100 may be implemented as a solution process-based metal-coated functional fiber. In detail, the metal-coated functional fiber may be implemented by forming a one-dimensional fibrous coating thin film through a continuous coating printing process and then performing heat treatment.

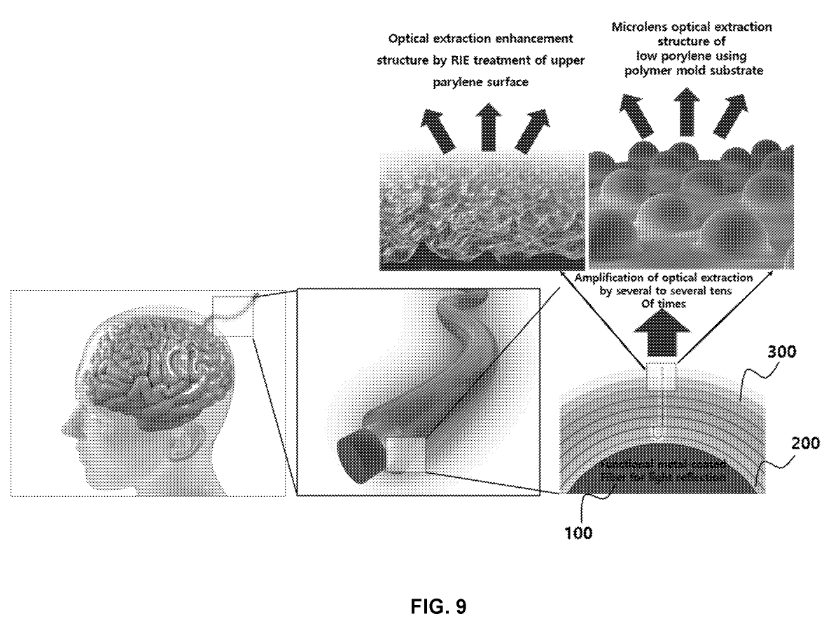

FIG. 9 is an exemplary view for explaining a light output structure in the electronic medicine according to an embodiment.

FIG. 9 illustrates an electronic medicine inserted into a brain to treat glioblastoma multiforme.

As illustrated in FIG. 9, due to overlapping of light of the transparent flexible OLED that has been wound multiple times and reflection of light traveling in a direction of the fiber, an optical output may be amplified by several tens of times compared to the related art to enable an ultra-high power operation. In addition, a surface of upper perylene may be subjected to reactive ion etching (RIE) treatment to improve light extraction, and a micro-lens light extraction structure of lower perylene using a polymer mold substrate may be proposed.

Thus, in the present disclosure, the implantable ultra-high power OLED fiber electronic medicine using the light reflection and overlapping may be proposed by repeatedly rolling the OLED having the transparent flexible properties in terms of materials and structures onto the metal-coated fiber.

In the present disclosure, the transparent element 300 may be implemented as a high-efficiency transparent flexible OLED.

In the present disclosure, since light is reflected again using the metal-coated fiber, the electronic medicine may be implemented as a transparent electrode capable of suppressing light absorption that occurs whenever the reflected light passes through an electrode layer of each transparent OLED.

In addition, the transparent flexible OLED proposed in the present disclosure may be designed to be manufactured with an ultra-thin symmetric structure of about 10 μm or less so that a neutral axis may be disposed within the OLED device, thereby preventing electrode cracking caused by the rolling and thus may utilize the transparent conductive oxide films (thin films of IZO and ITO).

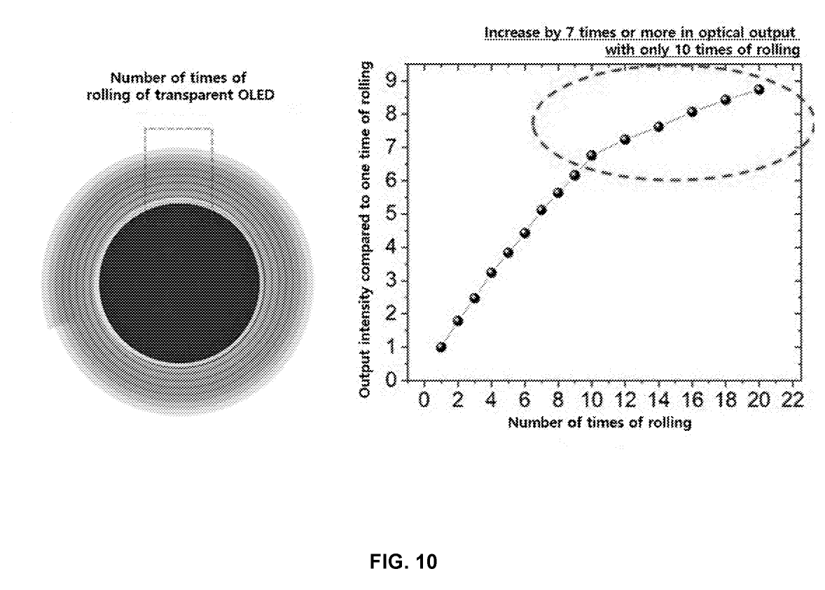

FIG. 10 is a graph showing results of simulating a change in light output depending on the number of times of rolling according to an embodiment.

Referring to FIG. 10, results of an optical computational simulation for predicting a change in light output according to the number of times of rolling of the transparent OLED fiber may be illustrated, and it may be confirmed that the light output increases by approximately 7 times to approximately 8 times. For example, when the number of times of rolling (number of times of folding) of the transparent OLED is about 10 times, it may be confirmed that the light output increases by at least 7 times.

According to the present disclosure, the electronic medicine may be manufactured by manufacturing the ultra-thin transparent flexible display element to roll the ultra-thin transparent flexible display element onto the fiber having the certain diameter, and thus, the light output intensity may increase depending on the number of times of rolling to enable the various biomedical applications through in vivo attachment and in vivo insertion that require the high power.

Although the present invention has been described using several preferred embodiments, these embodiments are illustrative and not limited thereto. Those skilled in the art will understand that various changes and modifications can be made without departing from the spirit of the present invention and the scope of the claims set forth in the appended claims.

### Description of the Symbols

100: Fiber 200: Adhesive

300: Light-emitting element 400: Encapsulation part

410: Inorganic layer 420: Polymer layer

430: Organic layer

What is claimed is:

1. An electronic medicine comprising:
a fiber;
a light-emitting element wound around the fiber; and
an encapsulation part, which is an encapsulation barrier configured to encapsulate a surface of the light-emitting element.

2. The electronic medicine of claim 1, wherein an adhesive is applied between the fiber and the light-emitting element.

3. The electronic medicine of claim 2, wherein the light-emitting element is provided in the form of a patch,
the adhesive is applied to one surface of the light-emitting element, and
the light-emitting element is wound around the fiber in a manner, in which the fiber is rolled from one end to the other end of the one surface of the light-emitting element, to which the adhesive is applied.

4. The electronic medicine of claim 1, wherein the light emitting-element comprises an organic light-emitting diode (OLED).

5. The electronic medicine of claim 1, wherein the encapsulation part comprises:
an inorganic layer disposed on the light-emitting element and made of an inorganic material;
a polymer layer disposed on the inorganic layer and made of a polymer material; and
an organic layer disposed on the polymer layer and made of an organic material.

6. The electronic medicine of claim 5, wherein the encapsulation layer is provided by crossing one or more inorganic layers and one or more polymer layers.

7. The electronic medicine of claim 5, wherein the organic layer is made of parylene-C.

8. A method for manufacturing an electronic medicine, the method comprising:
applying an adhesive to one surface of a patch-type light-emitting element;
positioning a fiber at one end of one surface of the light-emitting element, to which the adhesive is applied, to perform rolling while winding the fiber up to the other end of the one surface of the light-emitting element; and
forming an encapsulation part on the other surface of the light-emitting element.

9. The method of claim 8, wherein the light emitting-element comprises an organic light-emitting diode (OLED).

10. The method of claim 8, wherein the forming of the encapsulation part comprises:
forming an inorganic layer made of an inorganic material on the light-emitting element;
forming a polymer layer made of a polymer material on the inorganic layer; and
forming an organic layer made of an organic material on the polymer layer.

11. The method of claim 10, wherein, in the forming of the encapsulation part, one or more inorganic layers and one or more polymer layers are formed to cross each other.

12. The method of claim 10, wherein the organic layer is made of parylene-C.
